(19) United States
(12) Patent Application Publication
(10) Pub. No.: US 2026/0043052 A1
(43) Pub. Date: Feb. 12, 2026

(54) LIGNIN BINDER

(71) Applicant: STAM AGRO NV, Dendermonde (BE)

(72) Inventors: Ceder ALLOO, Dendermonde (BE); Jef VANOVERSCHELDE, Dendermonde (BE); Vaast VANOVERSCHELDE, Dendermonde (BE)

(21) Appl. No.: 19/102,564

(22) PCT Filed: Aug. 10, 2023
(86) PCT No.: PCT/IB2023/058089

§ 371 Date: Feb. 10, 2025

(30) Foreign Application Priority Data
Aug. 10, 2022 (BE) BE2022/5626

(51) Int. Cl. C12P 7/10; C12N 9/24

(52) U.S. Cl. CPC C12P 7/10; C12N 9/2402

(57) ABSTRACT

Method for preparing a fermentation product from a fermentable mixture comprising the steps of: (i) bringing the fermentable mixture, comprising lignocellulosic biomass. into contact with microorganisms, enzymes, or a combination thereof; (ii) allowing at least part of the fermentable mixture to be processed by the microorganisms, the enzymes, or the combination thereof, whereby the fermentation product is obtained; (iii) purifying the fermentation product, wherein the fermentable mixture comprises a keratin-containing material, a hydrolysate of a keratin-containing material, or a mixture thereof, in an amount of between 0.01 and 15 m %, expressed in total amount of keratin-containing material and hydrolysate of keratin-containing material in g per g of fermentable mixture. The invention also relates to the use of a keratin-containing material, a hydrolysate of keratin-containing material, or a combination thereof for the fermentation of a fermentable mixture, comprising lignocellulose, to bioethanol.

### TECHNICAL FIELD

The invention relates to a method for preparing a fermentation product from a fermentable mixture.

In a second aspect, the invention also relates to a use of a hydrolysate of keratin-containing material.

### PRIOR ART

Both bioethanol and biodiesel are mainly produced from biomass. The plant material used for ethanol is a form of sugar in Brazil and the EU, and corn in the US. For biodiesel, the primary source is rapeseed oil in Germany or soybean oil in the US. The reason these sources are used is that they are known and already cultivated, and because the sugar, starch or oil is relatively easy to extract and process into fuel.

In the long term, however, the use of food crops as fuel is not optimal. Food crops require good agricultural soils, abundant water and large amounts of energy in the form of farm machinery and fertilizers. In addition, sugar in Brazil is often grown on land that might otherwise be rainforest, further depleting an already dwindling natural resource. Competition for food inputs will only increase and in the event of food shortages fuel for vehicles would become expensive. In addition, fuel yields from these crops are so low that unrealistic amounts of land would be required to substantially or completely replace fossil fuels.

Plant-based lignocellulosic biomass represents a large, renewable source of potential starting materials for the production of a variety of chemicals, plastics, fuels and animal feeds. For example, lignocellulosic biomass feedstocks comprise cellulose, a polymer of glucose, which can be hydrolyzed to provide fermentable sugar for use in ethanol production.

Cellulose hydrolysis can be performed by acid or enzymatic hydrolysis (EH). In EH, a family of enzymes can be used that work together to hydrolyze glycosidic bonds in polymeric lignocellulose molecules. Most EH is done at a lignocellulosic fiber concentration (which can be referred to as a % K), between about 5-10%, to ensure good contact between the enzymes and the fibers. At higher fiber concentrations, the cellulose may swell to provide very thick mixtures that are difficult to handle (e.g., transfer from one reactor to another) and/or make it difficult to properly mix the enzymes and fibers, reducing the efficiency of the hydrolysis. Unfortunately, the low concentration of fiber during hydrolysis results in solutions containing low concentrations of simple sugars, increasing the size of the fermentation vessels that must be used during the ethanol production processes. A low sugar concentration also leads to a lower alcohol concentration after fermentation, requiring larger distillation columns and a higher energy input for the purification of fermented mixtures.

The conversion of plant biomass to ethanol via enzymatic cellulose hydrolysis offers a potentially sustainable route to biofuel production. However, the inhibition of enzymatic activity in pre-treated biomass by lignin severely limits the efficiency of this process. The cellulose surface is full. Nearly a quarter of the total cellulose surface is consistently covered by lignin, significantly reducing the area accessible to the enzymes. In addition, the presence of lignin molecules on the cellulose surface is likely to interfere with the mechanism of cellulose hydrolysis, reducing the distance a cellulose-bound enzyme can travel before its path is blocked by a lignin molecule.

Lignin exhibits an inhibitory effect during fermentation because the phenolic components of lignin reduce the efficiency of enzymatic hydrolysis, increasing the cost of ethanol production. For example, lignin directly and competitively inhibits the cellulase's recognition mechanism.

Thus, there is a need for efficient methods of fermenting lignocellulose. The methods are preferably suitable for achieving a high conversion at higher fiber concentrations (e.g. >10% K) and higher lignin concentrations. Such methods can be used to provide glucose and/or xylose solutions with a higher concentration, resulting in significant savings in capital and operating costs in alcohol production plants.

Methods are known in the literature for pretreating biomass with enzymes. Such a device is known, inter alia, from DK2880172 (DK ′172). DK ′172 discloses a method for treating biomass from lignin comprising: —providing a raw material for biomass from soft lignin cellulose, —pre-treating the raw material at a pH in the range of 3.5 to 9.0 in a one-step hydrothermal pre-treatment with a solids content of at least 35% at temperatures between 160 and 200° C. at residence times of less than 60 minutes, —separating the pre-treated biomass into a solid fraction and a liquid fraction, —hydrolyzing the solids fraction with or without addition of additional water content of 15% solids or higher over a period of 24 to 150 hours using enzymatic hydrolysis catalyzed by an enzyme mixture and-then mixing the separated liquid fraction and the hydrolyzed solid fraction after obtaining at least 50% conversion of cellulose into glucose, and carrying out further post-hydrolysis for a period of at least 6 hours, wherein xylo-oligomers in the liquid fraction are broken down into xylose monomers under the influence of residual enzyme activity in the hydrolyzed solid fraction.

This known method is complex, requires many enzymes and close monitoring. Moreover, this pretreatment has no effect on possible lignin inhibition. The present invention aims to solve at least some of the above problems or drawbacks. The object of the invention is to provide a method which partly obviates these drawbacks.

### SUMMARY OF THE INVENTION

In a first aspect, the present invention relates to a method according to claim 1. A hydrolysate is obtained from a keratin-containing material such as feathers, hair, hides, horn tissue, etc. The hydrolysate of keratin-containing material ensures higher yields of the fermentation product during fermentation, especially in the case of lignocellulosic biomass fermentation. Not only is more fermentation product formed, but it is also formed faster. The hydrolysate prevents inhibition by lignin, allowing fermentation to take place at a higher concentration of lignocellulosic biomass. As a result, less energy is needed during the fermentation and afterwards to purify the fermentation product. Expensive and polluting pre-treatment steps, such as using solvents to wash out the lignin, are not or less necessary due to the use of the hydrolysate.

Preferred embodiments of the method are set out in claims 2 up to and including 14.

In a second aspect, the present invention relates to a use according to claim 15.

This use has the advantage, among other things, that the problem of inhibition during fermentation of lignocellulosic biomass into bioethanol can be prevented.

It is an object of the invention to increase the yield of desired fermentation product, such as for instance bioethanol. This is done by reducing the inhibition of lignin during the fermentation of lignocellulosic biomass.

It is also an object of the invention to reduce the amount of enzymes and/or microorganisms required during fermentation.

### DETAILED DESCRIPTION

Unless otherwise defined, all terms used in the description of the invention, including technical and scientific terms, have the meaning as commonly understood by a person skilled in the art to which the invention pertains. For a better assessment of the description of the invention, the following terms are explained explicitly.

In this document, “a”, “an” and “the” refer to both the singular and the plural, unless the context clearly presupposes otherwise. For example, “a segment” means one or more than one segment.

Quoting numeric intervals by the endpoints includes all integers, fractions, and/or real numbers between the endpoints, including those endpoints.

The expression “percent by weight,” “wt %,” “m %” or “% by weight” in this document refers to the relative weight of a component based on the total weight of the entire referenced product.

“Biofuel” refers to fuel molecules (for example butanol, acetone and/or ethanol) produced from biomass preferably by microorganisms, for example in a microbial fermentation process.

“Biomass” refers to raw materials containing cellulose and/or starch such as: wood chips, corn cobs, rice, grasses, fodder crops, potatoes, tubers, roots, whole grain corn, grape pulp, cobs, grains, wheat, barley, rye, bran, grains, sugary raw materials (e.g. molasses, fruit, sugar cane or sugar beets), wood, pulp, by-products from the cardboard/paper processing industry, residues of sulfite waste liquor, and plant residues.

“Lignocellulosic” biomass refers to plant biomass containing cellulose, hemicelluloses and lignin. The carbohydrate polymers (cellulose and hemicelluloses) are entangled with lignin. “Lignins” are macromolecular components containing phenolic propylbenzene backbone units linked at various sites.

“Digestion” or “fermentation” can refer to the process in which an organic molecule is converted into another molecule with the help of a microorganism or enzymes. For example, fermentation can refer to transforming sugars or other molecules from biomass to produce alcohols (e.g., ethanol, methanol, butanol); organic acids (e.g. citric acid, acetic acid, itaconic acid, lactic acid, gluconic acid); ketones (e.g. acetone), amino acids (e.g. glutamic acid); gases (e.g. H₂ and CO₂), antibiotics (e.g. penicillin and tetracycline); enzymes; vitamins (e.g. riboflavin, B12, beta-carotene); and/or hormones. Thus, fermentation includes alcohol fermentation. For example, in another form, fermentation can refer to transforming amino acids into polyamines and/or biogenic amines. Fermentation comprises aerobic and anaerobic fermentations.

As used herein, the term “hydrolysate of keratin-containing material” includes all products of keratin-containing material prepared using an enzyme having appropriate proteolytic activity or acid hydrolysis or fermentation or thermal hydrolysis or a combination thereof. Commercially available hydrolysates can be used or hydrolysates can be prepared. By thermal hydrolysis is meant a process in which a mixture is heated under pressure to a temperature above its boiling point at 1 atmosphere.

In a first aspect, the invention relates to a method for preparing a fermentation product from a fermentable mixture.

According to an embodiment, the method comprises the steps of:
i. bringing the fermentable mixture, comprising lignocellulosic biomass, into contact with microorganisms, enzymes, or a mixture thereof;
ii. allowing at least part of the fermentable mixture to be processed by the microorganisms, the enzymes or a mixture thereof, whereby the fermentation product is obtained;
iii. purifying the fermentation product.

In a first step, the fermentable mixture is brought into contact with microorganisms, enzymes, or a combination thereof. Preferably, the fermentable mixture is brought into contact with enzymes. In a particularly preferred form, the enzymes comprise cellulases, hemicellulases or a combination thereof. A combination of cellulases and hemicellulases is advantageous. In another preferred form, the fermentable mixture is brought into contact with microorganisms, such as yeasts, molds, microbacteria, or a combination thereof.

The fermentable mixture comprises lignocellulosic biomass. Lignocellulosic biomass refers to plant dry matter (biomass), comprising carbohydrates and lignin. It is the most abundantly available feedstock on earth for the production of biofuels, mainly bioethanol. It is composed of carbohydrate polymers (cellulose, hemicellulose) and an aromatic polymer (lignin). These carbohydrate polymers contain different sugar monomers (six and five carbon sugars) and are tightly bound to lignin. Lignocellulosic biomass can be classified into virgin biomass, biomass waste and energy crops. Virgin biomass comprises all naturally occurring terrestrial plants such as trees, shrubs and grass. Waste biomass is produced as a low value by-product of various industrial sectors, such as agriculture (corn stover, sugar cane bagasse, straw, etc.) and forestry. Energy crops are crops with a high yield of lignocellulosic biomass, produced to serve as raw material for the production of second generation biofuel, for example from elephant grass.

According to a preferred form, the fermentable mixture comprises lignocellulosic biomass, in an amount of between 1.0 and 99 m % of the total fermentable mixture, preferably in an amount of between 1.0 and 50 m %, more preferably in an amount of between 1.0 and 40 m % lignocellulosic biomass, even more preferably in an amount between 1.0 and 25 m %, and most preferably in an amount between 1.0 and 15 m %.

In a preferred form, the fermentable mixture comprises a keratin-containing material, a hydrolysate of keratin-containing material, a derivative of keratin-containing material, or a mixture thereof. In a further preferred form, the fermentable mixture comprises a keratin-containing material, a hydrolysate of keratin-containing material, or a mixture thereof. The term “keratin-containing material” means material, preferably natural material, which consists of at least 10 m % keratin, preferably at least 20 m %, more preferably at least 30 m %, even more preferably at least 40 m %, even more preferably at least 50 m %, even more preferably at least 60 m %, even more preferably at least 70 m %, and most preferably at least 80 m %. The keratin may comprise alpha keratin, beta keratin, or a mixture thereof. Beta keratin is advantageous. Preferably, the keratin-containing material originates from birds, more preferably from chickens. The keratin-containing material may be in any form, for example, but not limited to, powder form.

In a specific preferred form, this keratin-containing material, a hydrolysate of keratin-containing material, a derivative of keratin-containing material, or a mixture thereof is added to the fermentable mixture comprising lignocellulosic biomass, and optionally the water, prior to step i. The keratin-containing material, a hydrolysate of keratin-containing material, a derivative of keratin-containing material, or a mixture thereof can thus be added as a fermentation enhancer. Throughout this specification, it should be understood that the addition of the keratin-containing material, a hydrolysate of keratin-containing material, a derivative of keratin-containing material, or a mixture thereof as an additive is preferred. The keratin-containing material, a hydrolysate of keratin-containing material, a derivative of keratin-containing material, or a mixture thereof preferably does not serve as a raw material for the fermentation since the raw material is the lignocellulosic biomass.

Keratin-containing material is for example, but not limited to, feathers, hair, hides, horn tissue, wool, etc., or a combination of these. Keratin-containing material is preferably selected from the list of: feathers, hair, hides, horn tissue, or products containing a high content of these substances, more preferably selected from the list of: feathers, hair or a mixture thereof. Particularly preferably, the keratin-containing material is feathers. “Horn tissue” is found in hooves, beaks, nails and the outer surface of horns.

A hydrolysate of keratin-containing material is, for example, but not limited to, a hydrolysate of feathers, a hydrolysate of hair, a hydrolysate of hides, a hydrolysate of horn tissue, or a mixture thereof. The hydrolysate of keratin-containing material is preferably chosen from the list of: a hydrolysate of feathers, a hydrolysate of hair, a hydrolysate of hides, a hydrolysate of horn tissue, a hydrolysate of residual products from the textile industry, or a mixture thereof, more preferably selected from the list of: hydrolysate of feathers, hydrolysate of hair, or a mixture thereof. Particularly preferably, the hydrolysate of keratin-containing material is a hydrolysate of feathers, also referred to as feather hydrolysate. Residual products from the textile industry include textile rags of animal origin, old woolen clothing, down residues or other keratin-containing material.

A derivative of keratin-containing material is, for example, but not limited to, a feather derivative, a hair derivative, a hide derivative, a horn tissue derivative, or a mixture thereof. For example, the term “derivative” is intended to mean acetylated, methylated, hydrogenated, hydrated, or glycosylated keratin-containing material.

In a preferred form, the fermentable mixture comprises a keratin-containing material, a hydrolysate of keratin-containing material, or a mixture thereof, in an amount of between 0.01 and 15 m %, expressed in total amount of keratin-containing material and hydrolysate of keratin-containing material in g per g of fermentable mixture, preferably in an amount between 0.01 and 10 m %, more preferably between 0.01 and 5.0 m %, even more preferably between 0.1 and 5.0 m %, even more preferably between 0.1 and 2.5 m %.

In a preferred form, the fermentable mixture comprises a keratin-containing material, a hydrolysate of keratin-containing material, or a mixture thereof, in an amount of between 0.01 and 15 m %, expressed in total amount of keratin-containing material and hydrolysate of keratin-containing material in g per g of fermentable mixture, preferably in an amount between 0.01 and 10 m %, more preferably between 0.01 and 5.0 m %, even more preferably between 0.01 and 1.0 m %, even more preferably between 0.05 and 0.5 m %, even more preferably between 0.1 and 0.5 m %.

In another or further preferred form, the fermentable mixture comprises a keratin-containing material, a hydrolysate of keratin-containing material, or a mixture thereof, in an amount of between 0.01 and 15 m %, expressed in total amount of keratin-containing material and hydrolysate of keratin-containing material in g per g of lignocellulosic biomass, preferably in an amount comprised between 0.1 and 10 m %, more preferably comprised between 1.0 and 10.0 m %, even more preferably comprised between 2.0 and 8.0 m %, and most preferably between 3.0 and 7.0 m %.

According to an embodiment, the fermentable mixture comprises a total amount of keratin-containing material and hydrolysate of keratin-containing material of 0.01-15 m %, expressed in total amount of keratin-containing material and hydrolysate of keratin-containing material in g per g of fermentable mixture. Preferably in an amount between 0.01 and 10 m %, more preferably between 0.01 and 5.0 m %, even more preferably between 0.01 and 1.0 m %, even more preferably between 0.05 and 0.5 m %, even more preferably between 0.1 and 0.5 m %.

When the fermentable mixture comprises keratin-containing material in the form of feathers, hair, hides, horn tissue, or a combination thereof, the keratin-containing material can be hydrolyzed during further processing by the microorganisms, the enzymes, acid, base or the combination thereof.

To this end, in an embodiment, an enzyme with suitable proteolytic activity, such as a keratinase, an acid or a base, can be added during the fermentation. Thus, the hydrolysis of the keratin-containing material can be promoted during the fermentation. Preferably, no additional additions need to be made.

In a preferred form, the fermentable mixture comprises a hydrolysate of a keratin-containing material, in an amount of between 0.01 and 15 m %, expressed in amount of hydrolysate of keratin-containing material in g per g of fermentable mixture, preferably in an amount between 0.01 and 10 m %, more preferably between 0.01 and 5.0 m %, even more preferably between 0.1 and 5.0 m %, even more preferably between 0.1 and 2.5 m %.

In a preferred form, the fermentable mixture comprises a hydrolysate of a keratin-containing material, in an amount of between 0.01 and 15 m %, expressed in amount of hydrolysate of keratin-containing material in g per g of fermentable mixture, preferably in an amount between 0.01 and 10 m %, more preferably between 0.01 and 5.0 m %, even more preferably between 0.01 and 1.0 m %, even more preferably between 0.05 and 0.5 m %.

In another or further preferred form, the fermentable mixture comprises a hydrolysate of a keratin-containing material, in an amount of between 0.1 and 15 m %, expressed in amount of hydrolysate of keratin-containing material in g per g of lignocellulosic biomass, preferably in an amount comprised between 0.1 and 10 m %, more preferably comprised between 1.0 and 10.0 m %, even more preferably comprised between 2.0 and 8.0 m %, and most preferably between 3.0 and 7.0 m %.

In a preferred form, the fermentable mixture comprises lignocellulosic biomass and a total amount of keratin-containing material and hydrolysate of keratin-containing material, in a mass ratio between 100/1 and 1/1, preferably in a mass ratio between 75/1 and 10/1, even more preferably a mass ratio between 60/1 and 10/1, preferably between 50/1 and 20/1.

In a preferred form, the fermentable mixture comprises lignocellulosic biomass and a total amount of keratin-containing material and hydrolysate of keratin-containing material, in a mass ratio of at least 100/1, preferably in a mass ratio of at least 75/1, even more preferably a mass ratio of at least 60/1, preferably a mass ratio of at least

When the fermentable mixture comprises a hydrolysate of keratin-containing material, the peptides present in the hydrolysate of keratin-containing material can partially bind the lignin. As a result, the inhibition by lignin is less great and a higher and faster conversion of the nutrients present in the fermentable mixture is obtained. The total amount of fermentation product produced is higher compared to a fermentation of a similar mixture without hydrolysate of keratin-containing material. The proteins or peptides present in the hydrolysate of keratin-containing material are extremely suitable for binding lignin and obtaining a better fermentation process.

The inventors found that the same, slightly less explicit, result is obtained with keratin-containing material. According to the inventors, the effect of the keratin-containing material can be attributed to the fact that the keratin-containing material is, however, hydrolyzed during the fermentation by, for example, microorganisms, the enzymes, acid, base or the combination thereof. The protein structures are broken down into peptides that are very suitable for interacting with lignin, just as is the case with the hydrolysate.

In a preferred form, the fermentable mixture is brought into contact with microorganisms, enzymes, or a combination thereof, in an amount of 0.01-10 m %, expressed in total amount of microorganisms and enzymes in g per g of lignocellulosic biomass. Preferably in an amount of 0.1-10 m %, more preferably 1-10 m %, even more preferably 2-8 m %.

In a particular embodiment, the fermentable mixture comprises keratin-containing material, and the fermentable mixture is pre-treated prior to step (i). According to this embodiment, the pretreatment can be done thermally, chemically or enzymatically.

The advantage of this embodiment is that the keratin-containing material will be hydrolyzed during the pre-treatment, as a result of which the lignin-binding effect will also be obtained.

According to an embodiment, the hydrolysate is obtained by thermal and/or enzymatic hydrolysis of keratin-containing material.

When the hydrolysate is obtained by thermal and/or enzymatic hydrolysis of keratin-containing material, the protein structures are broken down into peptides that are very suitable to interact with lignin. Also during fermentation, keratin-containing materials can be converted into smaller peptides that are suitable to interact with lignin.

Hydrolysates are preferably prepared via enzymatic hydrolysis by methods and processes well known in the art but may also be prepared by other known hydrolytic techniques, such as acid hydrolysis. Typical preparation methods comprise treating the keratin-containing material with a proteolytic enzyme in an aqueous medium, dispersing the protein from the keratin-containing material in water, and adjusting the pH with an acid or a base to the optimal pH range of the enzyme to be used. The proteolytic enzyme may be an endoprotease, an exoprotease, or a combination thereof. The enzyme is preferably added in an amount of 0.2-5%, more preferably 0.5-2% based on the substrate protein and the reaction is carried out at optimum temperature and pH for the time required, depending on the enzyme and the desired degree of hydrolysis. The reaction is typically terminated by heating the mixture to a temperature high enough to inactivate the enzyme. The reaction mixture can optionally be neutralized before or after the heating step by using a suitable acid, e.g. hydrochloric acid, or a base, e.g. sodium hydroxide. According to an embodiment, fractions with a different molecular mass are then separated, preferably a first fraction has a molecular mass of less than 1000 Da, a second fraction has a molecular mass between 1000 and 5000 and a third fraction has a molecular mass between 5000 Da and 10,000 Da, a fourth fraction has a molecular mass between 10,000 and 30,000, and a fifth fraction has a molecular mass greater than 30,000 Da. If the water-insoluble or slightly water-soluble fraction of the reaction mixture is desired, it can be obtained, for example, by centrifugation or filtration techniques to obtain what is referred to here as a protein hydrolysate suspension. The protein hydrolysate suspension can be used as such or as washed with water and/or dried. The process used for drying is not critical, for example freeze drying, spray drying or any other process known in the art which produces a powder, either directly or through a milling step, may be used. Dried and powdered protein hydrolysate is particularly preferred because it efficiently entraps lipids. Proteolytic enzymes which can be used for preparing the hydrolysates of the present invention comprise enzymes of plant, microbial and animal origin, including enzymes from genetically modified organisms, which hydrolyze proteins to hydrolysates under appropriate reaction conditions. According to an embodiment, the hydrolysate is obtained by enzymatic hydrolysis with proteolytic enzymes, preferably selected from pepsin, trypsin, pancreatin and papain. It is also possible to use different enzymes one after the other, e.g., pepsin and trypsin or pepsin and pancreatin.

According to an embodiment, the hydrolysate is obtained via a preparation method comprising the steps:
providing a composition comprising keratin-containing material and water,
expressing at least a portion of the water from the composition;
adding an aqueous liquid to the composition thereby forming a hydrolysable mixture;
thermally hydrolyzing the hydrolysable mixture, thereby obtaining a hydrolyzed mixture;
at least partially separating the solid and liquid phases from the thermally hydrolyzed mixture, resulting in a second mixture with a dry matter content that is less than the dry matter content of the thermally hydrolyzed mixture, and a third mixture with a higher dry matter content.

In a first step of the preparation method for obtaining the hydrolysate, a composition comprising keratin-containing material and water is provided.

According to certain embodiments of the present invention, the composition may be any by-product or waste material of biological origin, including in particular organic material, particularly derived from poultry, bovines, ovines, porcines, pets, birds, fur-bearing animals, and the like, usually obtained from a slaughterhouse or fat rendering plant (hereinafter referred to as “starting material”), preferably obtained from a poultry or pig slaughterhouse or poultry or pig fat rendering plant. Today, the common practice is that these by-products or waste materials of biological origin are poultry feathers or hair, transported by truck from a slaughterhouse to a conversion plant. According to an embodiment, the composition comprises feathers as the only solid product. According to an embodiment, the composition comprises hair, for example wool, as the only solid product. According to an embodiment, the composition comprises feathers and hair as the only solid products. According to an embodiment, the composition comprises animal hides. According to an embodiment, the composition substantially comprises chicken feathers and water. According to an embodiment, the composition comprises residual products from the processing of feathers.

Usually, the dry matter content of the starting material on arrival at the conversion plant is unknown due to the fact that the pre-treatments at the slaughterhouse are often an unknown factor. Consequently, upon arrival at the conversion plant, said starting material is preferably subjected to a pretreatment step. Said pre-treatment step may include notably but is not limited to: adding and/or removing water, cleaning, washing, sorting, defatting, cutting, milling, grinding, or any combination thereof.

Such pretreatment step may facilitate the handling of the starting material such as to render the same more easily removable and transportable. It also removes unwanted organic and inorganic material, thereby increasing the quality of the final product. It may also further improve the efficiency of further processing steps.

Preferably, the starting material is supplied to a feeder system, which may comprise at least one large compartment such as a bunker for storing the starting material, and/or a washing and/or transport system. In the feeder system, the starting material can be mixed with water to obtain the composition of the process of the present invention.

Such a feeder system may advantageously provide at the same time for the transport of the starting material, as well as washing and/or separating the starting material from unwanted organic material, such as blood, fat, or portions of the animal's body, and/or from unwanted inorganic material, such as plastic, metal parts, sand and rubber. Said feeder system may also advantageously allow to segregate thick packs of entangled starting material into smaller, transportable portions.

The feeder system, as mentioned above, typically uses commercially available devices, such as screw conveyors, pumps, and/or revolving or rotary screens. Impurities or undesired matter can be eliminated by means of sieves positioned below the screw conveyor channel. Alternatively the revolving screen is provided with perforations in the walls that allow the elimination of impurities while tumbling.

According to an embodiment, the dry matter content is at most 40 m %, more preferably at most 30 m %, even more preferably at most 25 m %.

According to an embodiment, the keratin-containing material absorbs at least part of the water.

When the starting material comprises an amount of water that exceeds the preferred amount of water, the washing and/or conveying system advantageously comprises a dewatering screw, with the aim of removing at least part of the water from the starting material to obtain the composition of the present invention.

In a second step of the preparation method for obtaining the hydrolysate, the invention provides for expressing at least part of the water of the composition.

Preferably, the dry matter content of the composition after expression is at least 32 m %, more preferably at least 34 m %, even more preferably at least 36 m %, more preferably at least 38 m %, and most preferably at least 40 m % relative to the total weight of the composition.

It should further be clear that the dry matter content of the composition after expression is preferably at most 58 m %, more preferably at most 56 m %, even more preferably at most 54 m %, more preferably at most 52 m %, and most preferably at most 50 m % relative to the total weight of the composition.

According to an embodiment of the process according to the present invention, the act of expressing the at least part of the water of the composition can be performed by using a force of gravity, in particular the compressive force of the keratin-containing material present in the composition. More specifically, the keratin-containing material present in the composition may be packed together in the form of at least one layer, the keratin-containing material in the upper portion of the at least one layer exerting pressure on the keratin-containing material located in the lower portions of the at least one layer, thereby expressing at least a portion of the water at least partially absorbed by the keratin-containing material. Alternatively, the at least part of the water can be removed from the composition by means of a decanter.

In a preferred embodiment of the process according to the present invention, the act of expressing the at least part of the water of the composition can be performed using a dewatering press, of which several models are available on the market. Due to the act of expressing at least part of the water, the keratin-containing material are mechanically dewatered and macerated as they are forcibly conveyed through the press. A screw conveyor or screw press that continuously compresses the material may be used. Such a device comprises an elongated, cylindrical barrel housing containing an axially disposed rotatable shaft for driving a feed worm to advance wet material through the barrel, and a plurality of knife bars protruding between the worm flights for cooperating with the latter to effect a compression process. The press barrel is typically made of stainless steel cage bars, which capture, drain, and compress feathers or hair.

Water is collected and squeezed out of the keratin-containing material and may be drained through a sewage pipe located at the bottom, or the water can flow through slots in the compression barrel of the screw press. Preferably, the collected water is returned to the feeder system.

According to an alternative embodiment, dewatering can be carried out by thermal drying, for example by passing through jacketed, steam-heated vessels.

According to certain embodiments of the process of the present invention, the composition after being pressed out is preferably ground before the third step.

Grinding of the composition after squeezing can be carried out with a grinding device, a mixer equipped with a shaft with one or more rotating discs, or any other device available on the market. Radial vanes on the disc's periphery may be used to blow out the feathers and hair present in the composition after expression at high velocity. The rotating discs preferably have a tooth pattern. These devices shred the wet feathers or wet hair present in the composition after expression. The advantage of grinding the keratin-containing material before the hydrolysis is that the composition after expression will be a more pulpy mixture, allowing easier hydrolysis at lower energy costs.

In a third step of the preparation method for obtaining the hydrolysate, an aqueous liquid is added to the composition after expression, as described above, to form the hydrolysable mixture, the hydrolysable mixture having a dry matter content of between 20 and 50 m %.

According to a preferred embodiment of the process according to the present invention, said hydrolysable mixture has a dry matter content of at most 48 m %, preferably at most 46 m %, more preferably at most 44 m %, even more preferably at most 42 m % and most preferably at most 40 m %, relative to the total weight of the hydrolysable mixture. It should further be understood that said hydrolysable mixture has a dry matter content of at least 22 m %, preferably at least 24 m %, more preferably at least 26 m %, even more preferably at least 28 m % and most preferably at least 30 m %, relative to the total weight of the hydrolysable mixture.

According to certain embodiments of the process according to the present invention, the aqueous liquid is tap water. According to certain embodiments of the process according to the present invention, the aqueous liquid is water from a water purification system.

Preferably, the aqueous liquid has a temperature which is at least 50° C., more preferably at least 60° C., even more preferably at least 70° C., more preferably at least 80° C., and most preferably at least 85° C.

According to an embodiment, the aqueous liquid is a liquid comprising water, furthermore enriched with at least amino acids, polypeptides and/or proteins, wherein in particular these proteins, polypeptides and/or amino acids are derived from keratin-containing material.

According to a more preferred embodiment of the process according to the present invention, said aqueous liquid is an enriched proteinaceous liquid which is obtained as a product stream after hydrolyzing the hydrolysable mixture, as explained in detail below.

In a fourth step of the preparation method for obtaining the hydrolysate, the hydrolysable mixture is thermally hydrolyzed.

According to an embodiment, the hydrolysis time of the hydrolysable mixture of the present invention is at least 3 minutes, preferably at least 4 minutes, and most preferably at least 5 minutes. Furthermore, it should be clear that the hydrolysis time is at most 25 minutes, preferably at most 20, preferably at most 19 minutes, more preferably at most 18 minutes, even more preferably at most 17 minutes, more preferably at most 16 minutes, and most preferably not more than 15 minutes.

According to an embodiment, the thermal hydrolysis continues for a period between 10 sec and 4 hours, preferably between 1 min and 20 min, even more preferably between 5 and 15 min.

According to an embodiment, the hydrolysis can take place in one or more reactors. According to an embodiment, the at least one reactor is operated at an elevated pressure of at least 3 bar, preferably at least 4 bar, more preferably at least 5 bar, and most preferably at least 6 bar. Furthermore, it should be clear that the at least one reactor is advantageously used at an elevated pressure of at most 12 bar, preferably at most 11 bar, more preferably at most 10 bar. Advantageously, the at least one reactor is operated at a temperature of at least 135° C., preferably at least 145° C., more preferably at least 155° C., even more preferably at least 165° C., and most preferably at least 175° C. It is further understood that the at least one reactor is advantageously operated at a temperature of at most 245° C., preferably at most 235° C., more preferably at most 225° C., even more preferably at most 215° C., more preferably at most 205° C., and most preferably at most 195° C. According to an embodiment, this hydrolysate is used as a lignin binder during fermentation.

In a fifth step of the preparation method for obtaining the hydrolysate, at least part of the solid and liquid phases of the thermally hydrolyzed mixture are separated from each other, resulting in a second mixture with a dry matter content that is less than the dry matter content of the thermally hydrolyzed mixture, and a third mixture with a higher dry matter content.

According to an embodiment, the preparation method for obtaining a hydrolysate from keratin-containing material further comprises the step of separating the hydrolyzed mixture, as described in detail above, into i) at least one portion in solid phase and ii) at least one liquid portion by means of suitable equipment. As typical suitable equipment mention may be made in particular of a press and a decanter and/or a centrifuge. In the context of the invention, the term “liquid portion” refers to the supernatant remaining on top of the solid phase portion in the suitable equipment used for the separation.

According to an embodiment, the dry matter content after removal of at least a part of the solid phase is between 5 and 30 m %, i.e. of the second mixture. The dry matter content in the remaining fraction, the third mixture, is higher than 30 m %.

According to an embodiment, at least part of the at least one liquid portion is subjected to evaporation, whereby an enriched proteinaceous liquid is formed, with a dry matter content. Preferably, the dry matter content of the enriched liquid is at least 40 m %, preferably at least 43 m %, more preferably at least 45 m %, and most preferably at least 48 m %. Furthermore, it should be clear that the dry matter content of the enriched liquid is at most 60 m %, preferably at most 57 m %, and more preferably at most 54 m %. It should be understood that the enriched liquid may be liquid at the vaporization temperature but may be a paste at room temperature.

Surprisingly, the inventors have now found that hydrolysis of keratin results in an increase in the content of low molecular weight protein and/or amino acids suitable for binding lignin. A lignin binder is a product that counteracts inhibition by lignin during fermentation, preferably only limited amounts are needed for this.

Because the mixture is hydrolyzed, the structure is partly broken down and opened. Because this thermal hydrolysis takes place in an aqueous environment, no special chemicals, acids or bases are required, nor a neutralization step. The thermal hydrolysis ensures a reduced cost over the entire process. Less expensive enzymes must be used to carry out the reaction. In addition, during thermal hydrolysis and/or during other steps, undesirable molecules, for example sugars or fats, are removed from the mixture. The mixture will also be rendered completely sterile by the thermal hydrolysis, preventing unwanted (bacterial) reactions that can influence the characteristics of the end product in an undesired and/or uncontrolled manner. Therefore, this thermal hydrolysis step is chosen for a good process.

According to an embodiment, the second and/or third mixture is further hydrolyzed enzymatically. According to an embodiment, endoproteases are used. According to an embodiment, exoproteases are used. According to an embodiment, exoproteases and endoproteases are used. According to an embodiment, the enzymatic hydrolysis takes place at a temperature higher than 40° C., preferably between 40 and 80° C., more preferably between 50 and 70° C. and even more preferably between 50 and 55° C. According to an embodiment, the enzymatic hydrolysis continues for a period of at least 10 minutes and preferably between 10 minutes and 12 hours, more preferably between 3 hours and 7 hours, even more preferably between 5 hours and 6 hours. According to an embodiment, the enzymatic hydrolysis takes place at a pH of 5 to 9, preferably between 6 and 8, preferably about 7.2. According to an embodiment, the water-to-protein ratio during enzymatic hydrolysis is between 50:1 and 1:1, preferably between 20:1 and 1:1. These parameters were found to be optimal for obtaining a hydrolysate suitable for lignin binding. An advantage over acid hydrolysis is that, for example, no neutralization is required afterwards and fewer salts are present. According to an embodiment, no strong acids or bases are added before or during the hydrolysis.

According to an embodiment of the preparation method according to the present invention, the preparation method further comprises a step of ultrafiltration, reverse osmosis and/or drying.

According to an embodiment, at least part of the hydrolysate is dried. Preferably, drying of at least part of the solid phase portion is suitably carried out on a band or belt dryer, a fluidized bed dryer, or a conveyor dryer. Preferably, the drying is carried out with air, the solid phase portion having a temperature of at most 80° C., preferably at most 75° C. and most preferably at most 70° C. According to an embodiment, the drying is carried out under atmospheric pressure, preferably at a negative pressure relative to the atmospheric pressure of −20 to −40 mbar, more preferably at a negative pressure of about-30 mbar.

According to an embodiment, at least part of the hydrolysate is subjected to ultrafiltration. The pressure during ultrafiltration is 2 to 10 bar and the membrane size is 2 nm to 0.1 μm. According to an embodiment, the pressure is between 2 and 5 bar and the membrane size is between 2 and 50 nm. According to an embodiment, the pressure is between 5 and 10 bar and the membrane size is between 50 and 100 nm.

According to an embodiment, at least part of the hydrolysate is subjected to reverse osmosis. The pressure during reverse osmosis is 10 to 100 bar and the membrane size is 0.1 nm to 1 nm. According to an embodiment, the pressure is between 10 and 50 bar and the membrane size is between 0.1 and 0.50 nm. According to an embodiment, the pressure is between 50 and 100 bar and the membrane size is between 0.5 and 1.0 nm.

According to an embodiment, at least a portion of the hydrolysate is subjected to a combination of at least two techniques including ultrafiltration, reverse osmosis and drying.

According to an embodiment, the preparation method further comprises a step of adding sulfur-binding components, preferably Cu₂(OH)₃Cl, ZnSO₄ of CuSO₄. These components ensure a reduction of the volatile sulfur components that can cause an undesirable odor.

According to another or further embodiment, the preparation method further comprises a removal step to remove unwanted components. In a preferred form, the removal step comprises a cation exchanger, an anion exchanger, an absorbent, an addition of metal salts, a nanofiltration, a microfiltration, or a combination thereof. Metal salts can react with sulfur and precipitate the compounds formed. The precipitated compounds can then be easily separated. According to a further embodiment, the preparation method comprises a removal step, in which undesirable components are removed via an addition of metal salts, nanofiltration, microfiltration or a combination thereof.

In addition, in an embodiment, the preparation method comprises sterilizing the hydrolysate to remove bacteria, particularly sulfite-reducing bacteria, sulfate-reducing bacteria, sulfur-reducing bacteria, or a combination thereof. The bacteria are often spore-forming bacteria. In a further preferred form, sterilization comprises heating to a temperature between 100 and 150° C., preferably between 110 and 140° C., more preferably between 120 and 130° C. Preferably, sterilization continues for a period of time between 5 and 30 minutes, more preferably between 10 and 25 minutes, even more preferably between 15 and 20 minutes. The sterilization is further carried out as known to a person skilled in the art. According to another embodiment of the preparation method, at least part of the liquid portion is used to form the aqueous liquid that is added to the composition, thereby forming the hydrolysable mixture as described above.

According to an alternative embodiment of the preparation method, the hydrolysate is mixed with at least part of the enriched liquid. According to an embodiment, the hydrolysate is mixed with an acid. Because an acid is added, the hydrolysate will remain stable for a longer period of time. According to an alternative embodiment of the preparation method, the hydrolysate is mixed with a stabilizing buffer or salt or organic acid. According to a further embodiment, the organic acid is a sorbate, phosphoric acid, lactate or formic acid.

According to an embodiment, an aqueous liquid comprising proteins and/or amino acids is added to the composition. This results in an increase in the protein and/or amino acid content of the hydrolysable mixture. As such, the protein and/or amino acids content of the final feather meal product can also be influenced.

The above-mentioned preparation method is a specific preparation method, from which it has been shown that a hydrolysate with an advantageous effect can be obtained. However, as mentioned above, a hydrolysate of keratin-containing material can also be prepared by any other known hydrolytic techniques, such as acid hydrolysis, enzymatic hydrolysis and thermal hydrolysis.

According to an embodiment, substantially no inorganic acid or base is used in the hydrolysate preparation method.

According to an embodiment, the fermentable mixture comprises 0.01-150 g lignin per kg dry matter, preferably 0.1-50 g and more preferably 0.3-10 g.

According to a preferred form, the fermentable mixture comprises 0.01-500 g lignin per kg dry matter, preferably 0.1-500 g lignin per kg dry matter, more preferably 1-500 g lignin per kg dry matter, even more preferably 1-350 g lignin per kg dry matter, even more preferably 50-350 g lignin per kg dry matter, even more preferably 100-350 g lignin per kg dry matter.

According to a preferred form, the lignocellulosic biomass comprises lignin, in an amount between 0.1 and 50 m %, preferably in an amount between 1 and 50 m %, more preferably in an amount between 1 and 40 m %, even more at preferably in an amount between 1 and 35 m %, even more preferably in an amount between 5 and 35 m %.

According to a preferred form, the fermentable mixture comprises lignin and a total amount of keratin-containing material and hydrolysate of keratin-containing material in a mass ratio of between 1 and 50, preferably between 1 and 25, even more preferably between 1 and 10.

According to an embodiment, the fermentable mixture comprises: lignocellulosic biomass, in an amount of 1.0-50 m %; a total amount of keratin-containing material and hydrolysate of keratin-containing material, in an amount of 0.01-15 m %; and water, in an amount of 40-98 m %. According to a further embodiment, the fermentable mixture comprises: lignocellulosic biomass, in an amount of 1.0-40 m %; a total amount of keratin-containing material and hydrolysate of keratin-containing material, in an amount of 0.01-10 m %; and water, in an amount of 55-98 m %.

According to an embodiment, the fermentable mixture comprises 10-80 m % water and 0.05-5 m % peptides from keratin-containing material. According to an embodiment, the fermentable mixture comprises 10-80 m % water, 15-40 m % dry lignocellulosic biomass and 0.05-5 m % peptides from keratin-containing material.

According to an embodiment, the lignocellulosic biomass is selected from the group consisting of herbaceous material, agricultural residues, forestry residues, municipal solid waste, waste paper, pulp and residues from the paper industry, or a combination thereof. According to an embodiment, the lignocellulosic biomass is selected from the group consisting of corn stover, straw, bagasse (from sugar cane or sorghum), sugar cane waste, wheat straw, rice straw, various types of sorghum, arundo, and other agricultural residues, miscanthus, switchgrass, bamboo, water hyacinth, wood chips, and wood pulp. According to an embodiment, the lignocellulosic biomass is ensiled. Ensiling is a method of preserving fodder crops and is a choice for stable storage of raw materials. During ensiling, the low pH caused by fermentation of free sugars inhibits microbes that break down polysaccharides and thereby reduces the breakdown of carbohydrates in the raw material. The silage may contain one or more strains of lactic acid bacteria. When they are close together, the supply of oxygen is limited and associated microorganisms will deplete the available oxygen. When access to oxygen is low, the available sugars will be fermented to acids (and sometimes alcohol) by accompanying fermenting microorganisms. According to an embodiment, the lignocellulosic biomass, measured on a dry matter basis, comprises less than 10 m % starch or sugars, preferably less than 5 m % and more preferably less than 3 m %. According to an embodiment, the lignocellulosic material comprises or consists of bagasse. Bagasse refers to the fibrous residue left after sugar cane or sorghum stalks are crushed to extract their juice. The bagasse may contain sugar left over from the extraction of the juice. According to an embodiment, fermentation takes place at a fiber concentration of 7-30% K, preferably 10-25% K.

According to an embodiment, the method further comprises pre-treating the lignocellulosic biomass to increase the enzymatic digestibility. According to an embodiment, the pretreatment comprises one or more of the group consisting of removing or modifying lignin, removing hemicellulose, decrystallizing cellulose, removing acetyl groups from hemicellulose, reducing the degree of polymerization of cellulose, increasing the pore volume of the lignocellulosic biomass and increasing the surface area of lignocellulose.

According to an embodiment, the pretreatment comprises one or more pretreatment techniques selected from the group consisting of autohydrolysis, steam explosion, milling, chopping, ball milling, compression milling, irradiation, flow-through treatment with liquid hot water, treatment with dilute acid, treatment with concentrated acid, treatment with peracetic acid, supercritical carbon dioxide treatment, alkali treatment, organic solvent treatment, cellulose solvent treatment and aerobic mold treatment. According to an embodiment, the alkali treatment is selected from the group consisting of sodium hydroxide treatment, lime treatment, wet oxidation, ammonia treatment, and oxidative alkali treatment. According to an embodiment, the alkali treatment is a green liquid treatment.

According to an embodiment, contacting the lignocellulosic biomass with the first enzyme composition comprises mixing the lignocellulosic biomass with the first enzyme composition at a solids concentration of about 5%. According to an embodiment, the first hydrolysis mixture comprises between about 5 filter paper units (FPU) and about 85 FPU of lignocellulose hydrolyzing enzyme per gram of lignocellulosic biomass. According to an embodiment, the first hydrolysis mixture comprises about 10 FPU of lignocellulose hydrolyzing enzyme per gram of lignocellulosic biomass. According to an embodiment, the first enzyme composition comprises cellulase. According to an embodiment, the first enzyme composition further comprises xylanase and P-glucosidase.

According to an embodiment, the fermentation product comprises methanol, ethanol, propanol, butanol, lactate, or a combination thereof. According to an embodiment, fermenting comprises contacting the fermentable mixture with a microorganism to provide an alcohol mixture and distilling the alcohol mixture to provide the alcohol. According to an embodiment, the microorganism is a yeast. According to an embodiment, the method further comprises dehydrating the alcohol to remove residual water. According to an embodiment, the alcohol is ethanol. According to an embodiment, the fermentation product comprises ethanol.

According to an embodiment, the keratin hydrolysate has the property that more than 25 m % of the peptides have a molecular mass higher than 1000 Dalton (Da), preferably more than 50 m %. Because the hydrolysate has the property that more than 25 m % of the peptides have a molecular mass higher than 1000 Dalton (Da), a significant proportion of the peptides is large enough to bind with the lignin over a long distance. As a result, several attachment sites are shielded. According to an embodiment, the hydrolysate has the property that more than 15 m % of the peptides have a molecular mass below 1000 Dalton, preferably more than 30 m %. Because the hydrolysate has the property that more than 15 m % of the peptides have a molecular mass lower than 1000 Dalton, preferably more than 30 m %, a significant proportion of the peptides is readily water-soluble. The peptides are small enough to nestle between the lignin and cellulose fibers and bind to the lignin. According to an embodiment, the hydrolysate has the property that 15-25 m % of the peptides have a molecular mass below 1000 Dalton, that 20-35 m % of the peptides have a molecular mass between 1000 and 5000 Dalton, that 20-35 m % of the peptides have a molecular mass between 5 and 10 kDa, and that 25-40 m % of the peptides have a molecular mass greater than 10 kDa.

According to an embodiment, the hydrolysate comprises at least 5 times more peptides than fats and comprises at least 5 times more peptides than carbohydrates. Because the hydrolysate comprises at least 5 times more peptides than fats and comprises at least 5 times more peptides than carbohydrates, the hydrolysate comprises many peptides on a dry matter basis, suitable for binding to lignin. According to an embodiment, the hydrolysate comprises 70-90 m % peptides, 5-10 m % fats, 1-5 m % ash and 3-15 m % water. According to an embodiment, the hydrolysate consists, on dry matter basis, of 75-94 m % peptides, 5-15 m % fats, 1-10 m % ash.

According to an embodiment, the hydrolysate has the property that Glycine (GLY) and Glutamic acid (GLU) comprise at least 10 m % of the amino acids present in the peptides. Because the hydrolysate has the property that GLY and GLU comprise at least 10 m % of the amino acids present in the peptides, peptides are obtained that are better suited to bind lignin. They are both relatively small amino acids. Glycine is important for muscles, skin, connective tissue, collagen and the central nervous system. Glycine is essential for the production of hormones including growth hormone and insulin.

According to an embodiment, the hydrolysate has the property that cysteine comprises at least 5 m % of the amino acids present in the peptides. The sulfur present in cysteine could possibly bind the lignin and thus prevent the microorganisms and/or enzymes from coming close to the lignin.

According to a particularly preferred embodiment, the method comprises the steps of:
i. bringing the fermentable mixture, comprising lignocellulosic biomass, into contact with microorganisms, enzymes, or a mixture thereof;
ii. allowing at least part of the fermentable mixture to be processed by the microorganisms, the enzymes, or the mixture thereof, whereby the fermentation product is obtained, wherein the fermentation product comprises glucose and/or ethanol;
iii. purifying the fermentation product;
wherein the fermentable mixture comprises lignocellulosic biomass and a total amount of keratin-containing material and hydrolysate of keratin-containing material, in a mass ratio between 100/1 and 1/1, preferably in a mass ratio between 75/1 and 10/1, even more preferably a mass ratio between 60/1 and 10/1, preferably between 50/1 and 20/1.

According to a particularly preferred embodiment, the method comprises the steps of:
i. bringing the fermentable mixture comprising lignocellulosic biomass into contact with enzymes;
ii. allowing the enzymes to process at least a portion of the fermentable mixture, whereby the fermentation product is obtained, wherein the fermentation product comprises glucose and/or ethanol;
iii. purifying the fermentation product,
wherein the fermentable mixture comprises lignocellulosic biomass and a total amount of keratin-containing material and hydrolysate of keratin-containing material, in a mass ratio between 100/1 and 1/1, preferably in a mass ratio between 75/1 and 10/1, even more preferably a mass ratio between 60/1 and 10/1, preferably between 50/1 and 20/1.

According to a particularly preferred embodiment, the method comprises the steps of:
i. bringing the fermentable mixture, comprising lignocellulosic biomass, into contact with enzymes, wherein the fermentable mixture comprises 0.1-500 g lignin per kg dry matter, more preferably 1-500 g lignin per kg dry matter, even more preferably 1-350 g lignin per kg dry matter, even more preferably 50-350 g lignin per kg dry matter, even more preferably 100-350 g lignin per kg dry matter;
ii. allowing the enzymes to process at least a portion of the fermentable mixture, whereby the fermentation product is obtained;
iii. purifying the fermentation product,
wherein the fermentable mixture comprises a keratin-containing material, a hydrolysate of a keratin-containing material, or a mixture thereof.

According to a particularly preferred embodiment, the method comprises the steps of:
i. bringing the fermentable mixture, comprising lignocellulose biomass, into contact with microorganisms, enzymes, or a mixture thereof, wherein the fermentable mixture comprises 0.1-500 g lignin per kg dry matter, more preferably 1-500 g lignin per kg dry matter, even more preferably 1-350 g lignin per kg dry matter, even more preferably 50-350 g lignin per kg dry matter, even more preferably 100-350 g lignin per kg dry matter;
ii. allowing at least part of the fermentable mixture to be processed by the microorganisms, the enzymes, or the mixture thereof, whereby the fermentation product is obtained, the fermentation product comprising glucose and/or ethanol;
iii. purifying the fermentation product,
wherein the fermentable mixture comprises a keratin-containing material, a hydrolysate of a keratin-containing material, or a mixture thereof.

According to a particularly preferred embodiment, the method comprises the steps of:
i. bringing the fermentable mixture comprising lignocellulosic biomass into contact with enzymes;
ii. allowing the enzymes to process at least a portion of the fermentable mixture, whereby the fermentation product is obtained;
iii. purifying the fermentation product,
wherein the fermentable mixture comprises a feather hydrolysate, in an amount of between 0.01 and 15 m %, expressed in g feather hydrolysate, per g fermentable mixture.

In a second aspect, the invention relates to a use of a hydrolyzed keratin-containing material, a hydrolysable keratin-containing material, or a combination thereof as described above, for preventing lignin inhibition during fermentation of lignocellulosic biomass to bioethanol.

The use of the hydrolysate during fermentation of lignocellulose to bioethanol, especially in fermentation of lignocellulosic biomass, leads to higher yields of the fermentation product. Not only is more fermentation product formed, but it is also formed faster. Because the hydrolysate can prevent the inhibition by lignin, the fermentation can take place at a higher concentration of lignocellulosic biomass. As a result, less energy is needed during the fermentation and afterwards to purify the fermentation product. Expensive and polluting pre-treatment steps, such as using solvents to wash out the lignin, are not necessary due to the use of the hydrolysate.

The invention will now be further explained on the basis of the following example, without however being limited to this.

### EXAMPLES

### Examples 1-13

Cardboard having a composition as shown in Table 1 was torn into small pieces by hand. Next, the cardboard was drenched in liquid nitrogen and cut into smaller pieces and dust using a robotic cutter.

TABLE 1
Cardboard composition

| | |
|---|---|
| Acid insoluble lignin (%) | 13.7 |
| Acid soluble lignin (%) | 0.19 |
| Total glucose concentration (g/kg) | 564 |
| Total xylose concentration (g/kg) | 112 |
| Dry matter (%) | 92.52 |
| Organic material (%) | 82.58 |
| Ash (%) | 9.93 |

Examples 1-13 concern enzymatic hydrolysis tests performed with cardboard as a substrate. The starting conditions for the tests are shown in Table 2. Enzymes in this example are a mixture of cellulases and hemicellulases.

The concentrations of enzyme and feather hydrolysate to be added were determined on the basis of the amount of cardboard present in the reaction medium, namely 7.5 g cardboard/100 g suspension. 7.5 g of pretreated cardboard placed in 250 ml autoclaved, baffled Erlenmeyer flasks. Demineralized water was added to a final reaction weight of 100 g after which the Erlenmeyer flasks were closed with a paper cap and mixed well. The pH of this suspension was adjusted to pH 5.0 using 2M H2SO4. After adding the different concentrations of feather hydrolysate (0, 2 and 4%), the Erlenmeyer flasks were shaken for 20 min (150 rpm) at 50° C. After this, the different concentrations of enzyme were added (1, 2, 4, 6%). After mixing briefly, a starter sample was taken. This 0.5 ml sample was boiled to inactivate the enzyme, centrifuged and stored at −20° C. until further analysis. The Erlenmeyer flasks were incubated at 50° C. while shaking (150 rpm). After different hydrolysis times, a 1 ml sample was taken for determination of the concentration of monosaccharides. These 1 mL samples were always boiled to inactivate the enzyme. After centrifugation of the boiled samples, they were stored at −20° C. until HPAEC-PAD analysis of the sugars.

TABLE 2

| | g/100 g suspension | | | |
|---|---|---|---|---|
| | | Demi | | Feather hydrolysate |
| | Cardboard | water | Enzymes | (VH) |
| Example 1 | 7.50 | 92.50 | 0.08 | 0.00 |
| Example 2 | 7.50 | 92.50 | 0.08 | 0.15 |
| Example 3 | 7.50 | 92.50 | 0.08 | 0.30 |
| Example 4 | 7.50 | 92.50 | 0.15 | 0.00 |
| Example 5 | 7.50 | 92.50 | 0.15 | 0.15 |
| Example 6 | 7.50 | 92.50 | 0.15 | 0.30 |
| Example 7 | 7.50 | 92.50 | 0.30 | 0.00 |
| Example 8 | 7.50 | 92.50 | 0.30 | 0.15 |
| Example 9 | 7.50 | 92.50 | 0.30 | 0.30 |
| Example 10 | 7.50 | 92.50 | 0.45 | 0.00 |
| Example 11 | 7.50 | 92.50 | 0.45 | 0.15 |
| Example 12 | 7.50 | 92.50 | 0.45 | 0.30 |
| Example 13 | 7.50 | 92.50 | 0.00 | 0.30 |

A total chemical hydrolysis of the cardboard resulted in 564 g glucose/kg and 112 g xylose/kg. By adding 7.5% cardboard to demineralized water, maximum concentrations of 42.3 g glucose/L and 8.4 g xylose/L can be obtained. Based on these values as well as the concentrations of glucose and xylose measured in the

Erlenmeyer flasks at the different hydrolysis times, conversions of lignocellulose to glucose and hemicellulose to xylose were determined.

### Lignocellulose Conversions to Glucose

Lignocellulose conversions between 10 and 63% were observed after 69 h. This corresponds to glucose concentrations of 3.9 g/L to 26 g/L. If the concentration of enzyme increases, the lignocellulose conversion also increases from 14% (Example 1) to 51% (Example 10).

At 1% enzyme added (example 3) and 2% enzyme added (Example 6), the lignocellulose conversion in the presence of 4% VH is greater (respectively, 33% and 45%) than the conversion at 0% VH, namely 14% (Example 1) and 16% (Example 4), respectively and greater than the conversion at 2% VH, namely 10% (Example 2) and 26% (Example 5), respectively.

When 4% enzyme was administered, the conversion is higher for the 4% VH (63%, Example 9) than for the 2% VH (46%, Example 8) and even much higher than for the 0% VH (35%, Example 7). At 6% enzyme added, the conversion (70%) at 2% and 4% VH (Example 11 and 12) over the entire hydrolysis time is greater than when 0% VH (Example 10) was added. In the condition without enzyme but with 4% VH (Example 13) a glucose concentration between 10.1 to 197.6 mg/L was measured. This corresponds to a negligible lignocellulose conversion of 0 to 0.5%.

### Hemicellulose Conversions to Xylose

The concentrations of liberated xylose increase as the concentration of enzyme increases. When the concentration of VH increases, the conversion of the hemicellulose also increases. Negligible xylose concentration (5 to 93.7 mg/L) and thus hemicellulose conversion was observed for the condition with 0% enzyme and 4% VH (Example 13). The hemicellulose conversion at 4% VH addition (Examples 3, 6, 9 and 12) was higher in all tests than at 2% VH addition (Examples 2, 5, 8 and 11), which in turn were higher conversions than at 0% VH addition (Examples 1, 4, 7 and 10).

As expected, higher enzyme concentrations also result in higher final conversions after 69 h. It is clear that the addition of the feather hydrolysate has a positive effect on the conversion of ligno-and hemicellulose in cardboard, especially at the lower enzyme concentrations.

### Examples 14 and 15

This example concerns the amino acid composition of feathers and a feather hydrolysate, as shown in Table 3. The different amino acids were reported based on the total protein present in the product.

TABLE 3

| | Feathers | Feather hydrolysate |
|---|---|---|
| | (Example 14) | (Example 15) |
| Arginine | 6.27 | 7.03 |
| Lysine | 2.59 | 2.14 |
| Alanine | 6.06 | 4.54 |
| Threonine | 4.32 | 4.78 |
| Glycine | 9.21 | 7.78 |
| Valine | 6.34 | 7.58 |
| Serine | 9.83 | 10.73 |
| Proline | 10.20 | 11.13 |
| Isoleucine | 4.14 | 4.78 |
| Leucine | 7.05 | 8.03 |
| Methionine | 0.86 | 0.65 |
| Histidine | 0.85 | 0.71 |
| Phenylalanine | 0.81 | 4.87 |
| Glutamic acid | 13.90 | 11.2 |
| Aspartate | 8.04 | 6.18 |
| Cysteine | 2.21 | 4.28 |
| Tyrosine | 2.39 | 2.92 |
| Tryptophan | 0.41 | 0.66 |

### Example 16

Non-hydrolyzed keratin-containing material, such as feathers, hair and beaks, was also tested according to the protocol described above. The keratin-containing material also appeared to have a positive effect on the conversion of ligno-and hemicellulose in cardboard, mainly at the lower enzyme concentrations, although the hydrolysate still had the most positive effect.

According to the inventors, the effect of the keratin-containing material can be attributed to the fact that the keratin-containing material is hydrolyzed during the fermentation by, for example, microorganisms, the enzymes, acid, base or the combination thereof. The protein structures are broken down into peptides that are very suitable for interacting with lignin, just as is the case with the hydrolysate.

1. A method for preparing a fermentation product from a fermentable mixture comprising the steps of:
bringing the fermentable mixture, comprising lignocellulosic biomass, into contact with microorganisms, enzymes, or a mixture thereof;
allowing at least part of the fermentable mixture to be processed by the microorganisms, the enzymes, or the mixture thereof, whereby the fermentation product is obtained;
purifying the fermentation product, characterized in that the fermentable mixture has a total amount of keratin-containing material and hydrolysate of keratin-containing material, in a mass ratio of between 100/1 and 1/1.

2. The method according to claim 1, characterized in that the keratin-containing material is chosen from the list of feathers, hair, hides, horn tissue, or a combination thereof.

3. The method according to any of the preceding claims claim 1, characterized in that the fermentation product comprises glucose in an amount between 1 and 50 g of glucose per liter of the fermentation product.

4. The method according to any of the preceding claims claim 1, characterized in that the microorganisms, enzymes, or a combination thereof are in an amount of 0.01-10 m %, expressed in total amount of microorganisms and enzymes in g per g fermentable mixture.

5. The method according to claim 1, characterized in that the fermentable mixture comprises lignocellulosic biomass and a total amount of keratin-containing material and hydrolysate of keratin-containing material, in a mass ratio between 60/1 and 10/1.

6. The method according to claim 1, characterized in that the fermentable mixture comprises a hydrolysate of a keratin-containing material, in an amount of between 0.01 and 15 m %, expressed as hydrolysate of keratin-containing material in g per g fermentable mixture.

7. The method according to claim 6, characterized in that the hydrolyzed keratin-containing material is obtained by thermal hydrolysis, enzymatic hydrolysis, or a combination thereof, of a keratin-containing material.

8. The method according to any of claim 6, characterized in that the hydrolysate is obtained from a preparation method comprising the steps:
providing a composition comprising keratin-containing material and water;
expressing at least a portion of the water from the composition;
adding an aqueous liquid to the composition, whereby a hydrolysable mixture is formed;
thermally hydrolyzing the hydrolysable mixture;
at least partially separating the solid and liquid phases from the thermally hydrolyzed mixture, as a result of which a second mixture is obtained with a dry matter content that is less than the dry matter content of the thermally hydrolyzed mixture, and a third mixture with a higher dry matter content, wherein the preparation method further comprises a step wherein the second and/or third mixture is hydrolyzed by enzymatic hydrolysis.

9. The method according to claim 8, characterized in that the composition substantially comprises feathers as keratin-containing material.

10. The method according to claim 1, characterized in that the fermentable mixture comprises 0.01-150 g lignin per kg dry matter.

11. The method according to claim 1, characterized in that the fermentable mixture comprises 10-80 m % water and 0.05-5 m % peptides from the keratin-containing material.

12. The method according to claim 1, characterized in that the fermentation product comprises ethanol.

13. The method according to claim 1, characterized in that the hydrolysate comprises at least 5 times more peptides than fats and at least 5 times more peptides than carbohydrates.

14. The method according to claim 1, characterized in that the hydrolysate has the property that cysteine comprises at least 5 m % of the amino acids present in the peptides.

15. The method according to claim 1, characterized in that the fermentable mixture comprises a keratin-containing material, a hydrolysate of a keratin-containing material, or a mixture thereof, in an amount of between 0.01 and 15 m %, expressed in total amount of keratin-containing material and hydrolysate of keratin-containing material in g per g of fermentable mixture.

16. The method according to claim 1, characterized in that the fermentation product comprises glucose and/or ethanol.

17. The method according to claim 1, characterized in that the fermentable mixture comprises lignin and a total amount of keratin-containing material and hydrolysate of keratin-containing material in a mass ratio between 1 and 50.

18. Use of a keratin-containing material, a hydrolysate of keratin-containing material, or a combination thereof for the fermentation of a fermentable mixture, comprising lignocellulose, to bioethanol.

19. The method according to claim 2, characterized in that the keratin-containing material is feathers.

20. The method according to claim 3, characterized in that the fermentation product comprises glucose in an amount between 5 and 30 g of glucose per liter of the fermentation product.
